(19) United States
(12) Patent Application Publication
(10) Pub. No.: US 2008/0268016 A1
(43) Pub. Date: Oct. 30, 2008

(54) Engineered Renal Tissue

(76) Inventors: Carrie H. Fang, Pittstown, NJ (US); Dhanuraj Shetty, Jersey City, NJ (US); Sridevi Dhanaraj, Raritan, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/107,290

(22) Filed: Apr. 22, 2008

Related U.S. Application Data
(60) Provisional application No. 61/000,297, filed on Apr. 24, 2007.

(51) Int. Cl. A61F 2/00; A61K 35/12; A61B 19/02

(52) U.S. Cl. 424/423; 424/93.7; 206/570

(57) ABSTRACT

Biocompatible tissue repair implant devices and their methods of use are provided for repairing a diseased kidney tissue. The present invention relates to methods of removing a portion of kidney tissue from a host or donor, mincing it, placing it on a bioresorbable scaffold, and implanting the scaffold into a defect site in a kidney of a host or patient for use in the treatment of degenerative kidney diseases. The compositions and methods provide a pluripotent milieu for the de-novo generation of renal tubular structures in the replacement of diseased kidney tissue. The processes and devices are useful in the treatment of medical conditions and diseases relating to the kidneys such as trauma, necrosis, and both acute and chronic forms of renal failure.

### FIELD OF THE INVENTION

The present invention relates to tissue-engineered kidneys and portions or specific sections thereof, and methods for their production and use.

### BACKGROUND

The kidney is a vital organ in mammals, responsible for fluid homeostasis, waste excretion, and hormone production. There are a variety of possible injuries and disorders including cancer, trauma, infection, inflammation and iatrogenic injuries or conditions that can lead to chronic disease or cause reduction or loss of function of a kidney. The incidence of chronic kidney disease in the United States has reached epidemic proportions, and a significant number of these patients will develop end-stage renal disease (ESRD), with glomerular filtration rates too low to sustain life. Dialysis is the major treatment modality for ESRD, but it has significant limitations in terms of morbidity, mortality, and cost. Allogenic kidney transplantation provides significant benefits in terms of mortality and is ultimately less costly, but is hampered by a severe shortage of available donor organs. Acute renal failure (ARF) is also quite common, having a mortality rate that ranges from 20 to 70%. For a number of reasons, including aggressive care of an older patient population, the mortality rate due to ARF has not changed over the past 20 years despite advances in technology and therapies.

Although kidney disease has a variety of individual types, they appear to converge into a few pathways of disease progression. The functional unit of the kidney is the nephron. There is a decrease in functioning nephrons with the progression of the disease; the remaining nephrons come under more stress to compensate for the functional loss, thereby increasing the probability of more nephron loss and thus creating a vicious cycle. Furthermore, unlike tissues such as bone or glandular epithelia which retain significant capacity for regeneration, it has generally been believed that new nephron units are not produced after birth, that the ability of the highly differentiated tissues and structures of the kidneys have limited reparative powers and, therefore, that mammals possess a number of nephron units that can only decline during post-natal life. There is an increasing interest in developing novel therapies for kidney disease, including artificial organs, genetic engineering, and tissue engineering.

Some current approaches can be found in the following publications. In U.S. Pat. No. 6,498,142 Sampath et. al. describe the treatment of chronic renal failure by the administration of morphogens including OP-1. In US Patent Application publication 2004/0167634 Atala et. al. describe a prosthetic kidney that relies on a porous membrane structure having an effluent channel. In US Patent Application publication 2006/0204441 Atala et. al. describe cell scaffold matrices with incorporated therapeutic agents coupled with nanoparticles. In US Patent Application publication 2005/0277576 Franco describes a method of treatment using a combination of growth factors and cell therapy that relies on a time-delay sequence of administration of these components. In US Patent Application publication 2003/0129751 Grikscheit et. al. describe tissue-engineered organs utilizing organoid units to seed a scaffold. Kim et al. (Biotech Letters 25: 1505-1508, 2003) describe removing renal segments from rats, mincing the tissue and then straining it through a 200 μm sieve to remove large fragments. It was then suspended in cell culture medium and then seeded onto biodegradable polymer scaffolds. Implantation into rats, showed a possibility of reconstituting renal structures in-vivo after 4 weeks. Still, there remains a need for effective ways to promote growth of portions or in certain cases, entire kidneys.

### BRIEF SUMMARY OF THE INVENTION

The present invention is directed to a device and methods for engineering renal structures for treatment of mammalian subjects at risk of chronic renal failure, or at risk of the need for renal replacement therapy. The present invention relies on the use of minced tissue to provide for the rapid augmentation of implantable scaffold materials for the regeneration of tissues. Samples are preferably obtained from a healthy region of a host tissue, minced, and then applied to the surfaces of an implantable scaffold to take advantage of the specific properties, growth factors, population of organ specific cells, and progenitor cells present in the specific tissue sample used to create the minced tissue. More preferably, separate samples are taken from the cortex and medulla regions of a mature host kidney, or obtained from the pronephros, mesonephros, or metanephros regions of an embryonic or early development stage allogenic donor kidney. The tissue samples are separately minced and separately applied to different regions or surfaces of an implantable scaffold and implanted into the host to provide for the regeneration of kidney tissue. One benefit of this approach is that it can be done intra-operatively because isolation and expansion of the cells are not necessary.

### BRIEF DESCRIPTION OF THE DRAWINGS

FIG. 1 is a hematoxylin-eosin stained section of tissue showing the newly formed nephron-like structures obtained in a biocompatible scaffold after 4 weeks of implantation in a SCID mouse.

FIG. 2 is a hematoxylin-eosin stained section of tissue showing the newly formed tubular structures obtained in a biocompatible scaffold after 4 weeks of implantation in a SCID mouse.

### DETAILED DESCRIPTION OF THE INVENTION

In order to more clearly and concisely point out the subject matter of the claimed invention, the following definitions are provided for specific terms used in the following written description and appended claims.

As used herein with respect to clinical indications, the term “chronic” means persisting for a period of at least three, and more preferably, at least six months.

As used herein, the terms “renal cortex”, “cortex”, “renal medulla”, and “medulla” have their common meanings as to the anatomy of the kidney as known to one of ordinary skill in the art, and as described in standard physiology textbooks (see for example Tortora and Grabowski, “Principles of Anatomy and Physiology”, 10th edition, (2003) John Wiley and Sons, Inc., and also Guyton and Hall, “Textbook of Medical Physiology”, 10th edition, (2000) W.B. Saunders Co.).

As used herein, the term “minced tissue” refers to a sample of biological tissue that has been chopped, ground, sliced, cut, worked into a paste or otherwise reduced in minimum particle size from the native tissue state to having particles no larger than from about 50 microns to about 1 mm in size, and more preferably from about 200 microns to about 1 mm. The minced tissue contains tissue fragments, clumps or clusters of cells, individual whole cells, and may also contain a portion of ruptured cells. The cells liberated from the disrupted tissue by mincing are able to migrate through the surrounding environment.

As used herein, the term “bioresorbable polymer” refers to one that will break down into small segments when exposed to moist body tissue. The segments are then either absorbed or excreted by the body, either in their native state or as metabolized derivatives of their native state. More particularly, the biodegraded segments do not elicit a permanent chronic foreign body reaction because no permanent residue of the segment is retained by the body. The terms “biodegradable”, “bioresorbable”, “absorbable”, bioabsorbable”, and “resorbable” are equivalent and may be used interchangeably.

As used herein, the term “scaffold” refers to a sheet, block, cube, cylinder, rod, disc, tube, or any shaped piece of biocompatible material or combination of biocompatible materials used to contain, carry, or deliver an amount of at least one bioactive agent upon implantation into a mammal. The scaffold can be made from biodegradable or non-biodegradable materials, or a combination of biodegradable and non-biodegradable materials, as well as woven, non-woven, or combinations of woven and non-woven materials. Furthermore, the scaffold can be shaped to the desired size and shape before use, so as to conform to a defect site.

As used herein, the term “polyglycolide” is understood to include polyglycolic acid. Further, the term “polylactide” is understood to include polymers of L-lactide, D-lactide, meso-lactide, blends thereof, and lactic acid polymers and copolymers in which other moieties are present in amounts less than 50 mole percent.

For the purposes of the present invention the terms “woven” and “nonwoven” as applied to medical textiles have their common meanings as understood by one of ordinary skill in the art. In addition, the nonwovens of the present invention preferably have a density of about 60-150 mg/cc, and more preferably from about 60-100 mg/cc, and a thickness of about 2-4 mm.

As used herein, the term “culture medium” has the common meaning as understood by one of ordinary skill in the art. Exemplary culture mediums include for example, but are not limited to, Dulbecco's modified eagle medium (DEM), Hank's balanced salt medium, Glasgow minimum essential medium, Ames medium, Click's medium, nutrient mixtures HAM F-10 and HAM F-12. The terms “culture medium” and “culture media” are equivalent and can be used interchangeably.

The present invention uses scaffold material with minced tissue to regenerate structural kidney tissue. The main function of the mincing is to decrease the barrier for cells to migrate from the tissue, without destroying the cells or removing other bioactive agents from the tissue. After mincing the cells are able to migrate easily out of the tissue and populate and reorganize throughout a scaffold upon implantation into a host The process of mincing can be performed by any convenient means and methods. For example, one can excise a piece of tissue from a suitable source using a biopsy sampler, scalpel, or other methods known in the art, place the excised tissue sample into a convenient suitable container such as a dish, pan or tray and repeatedly cut, slice, or chop the tissue into small pieces with a scalpel until the average tissue piece is less than about 1 cubic millimeter. Alternatively, the excised tissue could be placed into a mechanical homogenizing device, such as a blender to efficiently mince or chop the tissue into small pieces.

It is desirable that the minced tissue be used immediately after mincing, that is within about one hour after mincing, and more preferably within about fifteen minutes after mincing. It is also desirable that the minced tissue be used immediately without the removal or destruction of any native or added bioactive agents, such as could occur by rinsing, filtering, sieving, centrifugation, or other mechanical separation techniques, or by treatment with enzymes, oxidants, reductants, chelating agents, antibodies, and the like. Any steps that can be taken to reduce the dehydration and desiccation of the tissue during and after the mincing process are beneficial and contemplated by the present invention. For example, desiccation can be reduced by adding isotonic buffered saline solution to the tissue sample during the mincing process. This addition of saline solution is a hydrating process, and is not to be confused with rinsing the tissue, which would wash away beneficial components. The minced tissue could be placed in a covered container, or covered with a gauze pad moistened with saline solution prior to use to reduce desiccation and dehydration. It is also contemplated by the present invention to add other agents to augment the viability of the tissue sample during the handling, preparation, and implantation, including the addition of culture medium with or without autologous serum, glucose, oxygen, adenosine triphosphate (ATP), NADH, cell survival factors such as Growth Hormone Releasing Peptide-6 [an agonist of the ghrelin receptor, J. Neurochem, 2006, 99(3):839-49], anti-apoptotic agents such as cell permeable pentapeptide V5 (Diabetics, 2007, in press), and other metabolic agents necessary and favorable for cellular viability.

A variety of biocompatible polymers can be used to make the biocompatible tissue implants or scaffold devices according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring. In embodiments where the scaffold includes at least one synthetic polymer, suitable biocompatible synthetic polymers can include polymers selected from the group consisting of aliphatic polyesters, poly (amino acids), poly (propylene fumarate), copoly (ether-esters), polyalkylenes oxalates, polyamides, tyrosine derived polycarbonates, poly (iminocarbonates), polyorthoesters, polyoxaesters, polyamidoesters, polyoxaesters containing amine groups, poly (anhydrides), polyphosphazenes, and blends thereof. Suitable synthetic polymers for use in the present invention can also include biosynthetic polymers based on sequences found in collagen, elastin, thrombin, fibronectin, starches, poly (amino acid), gelatin, alginate, pectin, fibrin, oxidized cellulose, chitin, chitosan, tropoelastin, hyaluronic acid, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of lactide (which includes lactic acid, D-, L-and meso lactide); glycolide (including glycolic acid); ε-caprolactone; p-dioxanone (1,4-dioxan-2-one); trimethylene carbonate (1,3-dioxan-2-one); alkyl derivatives of trimethylene carbonate; δ-valerolactone; β-butyrolactone; y-butyrolactone; ε-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; α, α diethyipropiolactone; ethylene carbonate; ethylene oxalate; 3-methyl-1,4-dioxane-2,5-dione; 3,3-diethyl-1,4-dioxan-2,5-dione; 6,6-dimethyldioxepan-2-one; 6,8-dioxabicycloctane-7-one and polymer blends thereof. Aliphatic polyesters used in the present invention can be homopolymers or copolymers (random, block, segmented, tapered blocks, graft, triblock, etc.) having a linear, branched or star structure. Poly (iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly (ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol, 30(1), page 498, 1989 by Cohn (e.g., PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes include co-, ter-, and higher order mixed monomer based polymers made from L-lactide, D, L-lactide, lactic acid, glycolide, glycolic acid, para-dioxanone, trimethylene carbonate and

ε-caprolactone, such as are described by Allcock in The Encyclopedia of Polymer Science, Vol. 13, pages 31-41, Wiley Intersciences, John Wiley & Sons, 1988 and by Vandorpe, et. al. in the Handbook of Biodegradable Polymers, edited by Domb, et al., Hardwood Academic Press, pp. 161-182 (1997). Polyanhydrides include those derived from diacids of the form HOOC—C6H4-O—(CH2),—O—C6H4-COOH, where “m” is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters include those such as described by Heller in Handbook of Biodegradable Polymers, edited by Domb, et al., Hardwood Academic Press, pp. 99-118 (1997).

Elastomeric copolymers are also particularly useful in the present invention. Suitable elastomeric polymers include those with an inherent viscosity in the range of about 1.2 dLg to 4 dLg, more preferably about 1.2 dLg to 2 dLg and most preferably about 1.4 dLg to 2 dLg as determined at 25° C. in a 0.1 gram per deciliter (g/dL) solution of polymer in hexafluoroisopropanol (HFIP).

Exemplary biocompatible elastomers that can be used in the present invention include, but are not limited to, elastomeric copolymers of ε-caprolactone and glycolide (including polyglycolic acid) with a mole ratio of ε-caprolactone to glycolide of from about 35:65 to about 65:35, more preferably from 35:65 to 45:55; elastomeric copolymers of ε-caprolactone and lactide (including L-lactide, D-lactide, blends thereof, and lactic acid polymers and copolymers) where the mole ratio of ε-caprolactone to lactide is from about 35:65 to about 65:35 and more preferably from about 30:70 to 45:55; other preferable blends include a mole ratio of ε-caprolactone to lactide from about 85:15 to 95:5; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40:60 to about 60:40; elastomeric copolymers of ε-caprolactone and p-dioxanone where the mole ratio of ε-caprolactone to p-dioxanone is from about from 30:70 to about 70:30; elastomeric copolymers of p-dioxanone and trimethylene carbonate where the mole ratio of p-dioxanone to trimethylene carbonate is from about 30:70 to about 70:30; elastomeric copolymers of trimethylene carbonate and glycolide (including polyglycolic acid) where the mole ratio of trimethylene carbonate to glycolide is from about 30:70 to about 70;30; elastomeric copolymers of trimethylene carbonate and lactide (including L-lactide, D-lactide, blends thereof, and lactic acid polymers and copolymers) where the mole ratio of trimethylene carbonate to lactide is from about 30:70 to about 70;30; and blends thereof. Examples of suitable biocompatible elastomers are described in U.S. Pat. Nos. 4,045,418; 4,057,537 and 5,468,253.

In one embodiment, the elastomer is a copolymer of 35:65 ε-caprolactone and glycolide, formed in a dioxane solvent and including a polydioxanone mesh. In another embodiment, the elastomer is a copolymer of 40:60 ε-caprolactone and lactide with a polydioxanone mesh. In yet another embodiment, the elastomer is a 50:50 blend of a 35:65 copolymer of ε-caprolactone and glycolide and 40:60 copolymer of ε-caprolactone and lactide. The polydioxanone mesh may be in the form of a one layer thick two-dimensional mesh or a multi-layer thick three-dimensional mesh.

The scaffold of the present invention can, optionally, be formed from a bioresorbable material that has the ability to resorb in a timely fashion in the body environment, that is the scaffold does not resorb so quickly that the body has not had sufficient time to incorporate new tissue growth into the scaffold, and also that the scaffold does not resorb so slowly as to be considered a semi-permanent implant. Thus, a preferable range of resorption time would be from about 2 weeks to about one year, and more preferably from about 4 weeks to about 6 months. The differences in the absorption time under in vivo conditions can also be the basis for combining two different copolymers when forming the scaffolds of the present invention. For example, a copolymer of 35:65 ε-caprolactone and glycolide (a relatively fast absorbing polymer) can be blended with 40:60 ε-caprolactone and L-lactide copolymer (a relatively slow absorbing polymer) to form a biocompatible scaffold. Depending upon the processing technique used, the two constituents can be either randomly inter-connected bicontinuous phases, or the constituents could have a gradient-like architecture in the form of a laminate type composite with a well integrated interface between the two constituent layers. The microstructure of these scaffolds can thus be optimized to facilitate regeneration or repair of the desired anatomical features of the tissue that is being repaired.

In one embodiment, it is desirable to use polymer blends to form scaffolds which transition from one composition to another composition in a gradient-like architecture. Clearly, one of ordinary skill in the art will appreciate that other polymer blends may be used for similar gradient effects, or to provide different gradients (e.g., different absorption profiles, stress response profiles, or different degrees of elasticity). For example, such design features can establish a concentration gradient for the suspension of minced tissue associated with the scaffolds of the present invention, such that a higher concentration of the tissue fragments is present in one region of the implant (e.g., an interior portion) than in another region (e.g., outer portions).

The biocompatible scaffold of the tissue repair implant of the present invention can also include a reinforcing material comprised of any absorbable or non-absorbable textile having, for example, knitted, warped knitted (i.e., lace-like), woven, non-woven, and braided structures. In one embodiment, the reinforcing material has a mesh-like structure. In any of the above structures, mechanical properties of the material can be altered by changing the density or texture of the material, the type of knit or weave of the material, the thickness of the material, or by embedding particles in the material. The mechanical properties of the material may also be altered by creating sites within the mesh where the fibers are physically bonded with each other or physically bonded with another agent, such as, for example, an adhesive or a polymer. The fibers used to make the reinforcing component can be monofilaments, yarns, threads, braids, or bundles of fibers. These fibers can be made of any biocompatible material including bioabsorbable materials such as polylactic acid (PLA), polyglycolic acid (PGA), polycaprolactone (PCL), polydioxanone (PDO), trimethylene carbonate (TMC), and copolymers or blends thereof. These fibers can also be made from any biocompatible materials based on natural polymers including silk and collagen-based materials. These fibers can also be made of any biocompatible fiber that is nonresorbable, such as, for example, polyethylene, polyethylene terephthalate, poly (tetrafluoroethylene), polycarbonate, polypropylene and polyvinyl alcohol. In one embodiment, the fibers are formed from a 90:10 copolymer of glycolide and lactide.

In another embodiment, the fibers that form the reinforcing material can be made of a bioresorbable glass. Bioglass, a silicate containing calcium phosphate glass, or calcium phosphate glass with varying amounts of solid particles added to control resorption time are examples of materials that could be spun into glass fibers and used for the reinforcing material. Suitable solid particles that may be added include iron, magnesium, sodium, potassium, and combinations thereof.

The biocompatible scaffolds as well as the reinforcing material may also be formed from a thin elastomeric sheet with pores or perforations to allow tissue ingrowth. Such a sheet could be made of blends or copolymers of polylactic acid (PLA), polyglycolic acid (PGA), polycaprolactone (PCL), and polydioxanone (PDO).

In one embodiment, filaments that form the biocompatible scaffolds or the reinforcing material may be co-extruded to produce a filament with a sheath/core construction. Such filaments are comprised of a sheath of biodegradable polymer that surrounds one or more cores comprised of another biodegradable polymer. Filaments with a fast-absorbing sheath surrounding a slower-absorbing core may be desirable in instances where extended support is necessary for tissue ingrowth.

One of ordinary skill in the art will appreciate that one or more layers of the reinforcing material may be used to reinforce the tissue implant of the invention. In addition, biodegradable textile scaffolds, such as, for example, meshes, of the same structure and chemistry or of different structures and chemistries can be overlaid on top of one another to fabricate biocompatible tissue implants with superior mechanical strength.

In embodiments where the scaffold includes at least one natural polymer, suitable examples of natural polymers include, but are not limited to, fibrin-based materials, collagen-based materials, hyaluronic acid-based materials, glycoprotein-based materials, cellulose-based materials, silks and combinations thereof. By way of nonlimiting example, the biocompatible scaffold can be constructed from a collagen-based small intestine submucosa.

In yet another embodiment of the tissue implants of the present invention, the scaffold can be formed using tissue grafts, such as may be obtained from autogeneic tissue, allogeneic tissue and xenogeneic tissue. By way of non-limiting example, tissues such as skin, cartilage, ligament, tendon, periosteum, perichondrium, synovium, fascia, mesenter and sinew can be used as tissue grafts to form the biocompatible scaffold. In some embodiments where an allogenic tissue is used, tissue from a fetus or newborn can be used to avoid the immunogenicity associated with some adult tissues.

In another embodiment, the scaffold could be in the form of an injectable gel that would cure in place at the defect site. The gel can be a biological or synthetic hydrogel, including alginate, cross-linked alginate, hyaluronic acid, collagen gel, fibrin glue, fibrin clot, poly (N-isopropylacrylamide), agarose, chitin, chitosan, cellulose, polysaccharides, poly (oxyalkylene), a copolymer of poly (ethylene oxide)-poly (propylene oxide), poly (vinyl alcohol), polyacrylate, platelet rich plasma (PRP) clot, platelet poor plasma (PPP) clot, MATRIGEL, or blends thereof.

In still yet another embodiment of the tissue implants, the scaffold can be formed from a polymeric foam component having pores with an open cell pore structure. The pore size can vary, but preferably, the pores are sized to allow tissue ingrowth. More preferably, the pore size is in the range of about 50 to 1000 microns, and even more preferably, in the range of about 50 to 500 microns. The polymeric foam component can, optionally, contain a reinforcing component, such as for example, the textiles disclosed above. In some embodiments where the polymeric foam component contains a reinforcing component, the foam component can be integrated with the reinforcing component such that the pores of the foam component penetrate the mesh of the reinforcing component and interlock with the reinforcing component.

The foam component of the tissue implant may be formed as a foam by a variety of techniques well known to those having ordinary skill in the art. For example, the polymeric starting materials may be foamed by lyophilization, supercritical solvent foaming (i.e., as described in EP 464,163), gas injection extrusion, gas injection molding, or casting with an extractable material (e.g., salts, sugars, or similar suitable extractable materials).

In one embodiment, the foam component of the engineered tissue repair implant devices may be made by a polymer-solvent phase separation technique, such as lyophilization. Generally, however, a polymer solution can be separated into two phases by any one of the following four techniques: (a) thermally induced gelation/crystallization; (b) non-solvent induced separation of solvent and polymer phases; (c) chemically induced phase separation, and (d) thermally induced spinodal decomposition. The polymer solution is separated in a controlled manner into either two distinct phases or two bicontinuous phases. Subsequent removal of the solvent phase usually leaves a porous structure with a density less than the bulk polymer and pores in the micrometer ranges. See for example “Microcellular Foams Via Phase Separation”, J. Vac. Sci. Technol., A. T. Young, Vol. 4(3), May/June 1986.

Suitable solvents that may be used in the preparation of the foam component include, but are not limited to, formic acid, ethyl formate, acetic acid, hexafluoroisopropanol (HFIP), cyclic ethers (e.g., tetrahydrofuran (THF), dimethylene fluoride (DMF), and polydioxanone (PDO)), acetone, acetates of C2 to C5 alcohols (e.g., ethyl acetate and t-butylacetate), glyme (e.g., monoglyme, ethyl glyme, diglyme, ethyl diglyme, triglyme, butyl diglyme and tetraglyme), methylethyl ketone, dipropyleneglycol methyl ether, lactones (e.g., Y-valerolactone, δ-valerolactone, β-butyrolactone, y-butyrolactone), 1,4-dioxane, 1,3-dioxolane, 1,3-dioxolane-2-one (ethylene carbonate), dimethlycarbonate, benzene, toluene, benzyl alcohol, p-xylene, naphthalene, tetrahydrofuran, N-methyl pyrrolidone, dimethylformamide, chloroform, 1,2-dichloromethane, morpholine, dimethylsulfoxide, hexafluoroacetone sesquihydrate (HFAS), anisole and mixtures thereof. Among these solvents, a preferred solvent is 1,4-dioxane. A homogeneous solution of the polymer in the solvent is prepared using standard techniques.

The applicable polymer concentration or amount of solvent that may be utilized will vary with each system. Generally, the amount of polymer in the solution can vary from about 0.5% to about 90% and, preferably, will vary from about 0.5% to about 30% by weight, depending on factors such as the solubility of the polymer in a given solvent and the final properties desired in the foam.

In one embodiment, solids may be added to the polymer-solvent system to modify the composition of the resulting foam surfaces. As the added particles settle out of solution to the bottom surface, regions will be created that will have the composition of the added solids, not the foamed polymeric material. Alternatively, the added solids may be more concentrated in desired regions (i.e., near the top, sides, or bottom) of the resulting tissue implant, thus causing compositional changes in all such regions. For example, concentration of solids in selected locations can be accomplished by adding metallic solids to a solution placed in a mold made of a magnetic material, or by adding magnetic solids to a solution placed in a mold made of a metallic material.

A variety of types of solids can be added to the polymer-solvent system. Preferably, the solids are of a type that will not react with the polymer or the solvent. Generally, the added solids have an average diameter of less than about 1.0 mm and preferably will have an average diameter of about 50 to about 500 microns. Preferably, the solids are present in an amount such that they will constitute from about 1 to about 50 volume percent of the total volume of the particle and polymer-solvent mixture (wherein the total volume percent equals 100 volume percent).

Exemplary solids include, but are not limited to, particles of demineralized bone, calcium phosphate particles, bioglass particles, calcium sulfate, or calcium carbonate particles for bone repair, leachable solids for pore creation and particles of bioabsorbable polymers not soluble in the solvent system that are effective as reinforcing materials or to create pores as they are absorbed, and non-bioabsorbable materials.

Suitable leachable solids include nontoxic leachable materials such as salts (e.g., sodium chloride, potassium chloride, calcium chloride, sodium tartrate, sodium citrate, and the like), biocompatible mono and disaccharides (e.g., glucose, fructose, dextrose, maltose, lactose and sucrose), polysaccharides (e.g., starch, alginate, chitosan), and water soluble proteins (e.g., gelatin and agarose). The leachable materials can be removed by immersing the foam with the leachable material in a solvent in which the particle is soluble for a sufficient amount of time to allow leaching of substantially all of the particles, but which does not dissolve or detrimentally alter the foam. The preferred extraction solvent is water, most preferably distilled-deionized water. Such a process is described in U.S. Pat. No. 5,514,378. Preferably the foam will be dried after the leaching process is complete at low temperature and/or vacuum to minimize hydrolysis of the foam unless accelerated absorption of the foam is desired.

Suitable non-bioabsorbable materials include biocompatible metals such as stainless steel, cobalt chrome, titanium and titanium alloys, and bioinert ceramic particles (e.g., alumina, zirconia, and calcium sulfate particles). Further, the non-bioabsorbable materials may include polymers such as polyethylene, polyvinylacetate, polymethyl methacrylate, polypropylene, poly (ethylene terephthalate), silicone, polyethylene oxide, polyethylene glycol, polyurethanes, polyvinyl alcohol, natural polymers (e.g., cellulose particles, chitin, and keratin), and fluorinated polymers and copolymers (e.g., polyvinylidene fluoride, polytetrafluoroethylene, and hexafluoropropylene).

It is also possible to add solids (e.g., barium sulfate) that will render the tissue implants radio opaque. The solids that may be added also include those that will promote tissue regeneration or regrowth, as well as those that act as buffers, reinforcing materials or porosity modifiers.

As noted above, porous, reinforced tissue repair implant devices of the present invention are made by injecting, pouring, or otherwise placing the appropriate polymer solution into a mold set-up comprised of a mold and the reinforcing elements. The mold set-up is then cooled in an appropriate bath or on a refrigerated shelf and then lyophilized, thereby providing a reinforced scaffold. A biological component can be added either before or after the lyophilization step. In the course of forming the foam component, it is believed to be important to control the rate of freezing of the polymer-solvent system. The type of pore morphology that is developed during the freezing step is a function of factors such as the solution thermodynamics, freezing rate, temperature to which it is cooled, concentration of the solution, and whether homogeneous or heterogeneous nucleation occurs. One of ordinary skill in the art can readily optimize the parameters without undue experimentation.

The required general processing steps include the selection of the appropriate materials from which the polymeric foam and the reinforcing components are made. If a mesh reinforcing material is used, the proper mesh density must be selected. Further, the reinforcing material must be properly aligned in the mold, the polymer solution must be added at an appropriate rate and, preferably, into a mold that is tilted at an appropriate angle to avoid the formation of air bubbles, and the polymer solution must be lyophilized.

In embodiments that utilize a mesh reinforcing material, the reinforcing mesh should be of a certain density. That is, the openings in the mesh material must be sufficiently small to render the construct sutureable or otherwise fastenable, but not so small as to impede proper bonding between the foam and the reinforcing mesh as the foam material and the open cells and cell walls thereof penetrate the mesh openings. Without proper bonding the integrity of the layered structure is compromised, leaving the construct fragile and difficult to handle. Because the density of the mesh determines the mechanical strength of the construct, the density of the mesh can vary according to the desired use for tissue repair. In addition, the type of weave used in the mesh can determine the directionality of the mechanical strength of the construct, as well as the mechanical properties of the reinforcing material, such as for example, the elasticity, stiffness, burst strength, suture retention strength and ultimate tensile strength of the construct. By way of non-limiting example, the mesh reinforcing material in a foam-based biocompatible scaffold of the present invention can be designed to be stiff in one direction, yet elastic in another, or alternatively, the mesh reinforcing material can be made isotropic.

During the lyophilization of the reinforced foam, several parameters and procedures are important to produce implants with the desired integrity and, mechanical properties. Preferably, the reinforcing material is substantially flat when placed in the mold. To ensure the proper degree of flatness, the reinforcing material (e.g. mesh) is pressed flat using a heated press prior to its placement within the mold. Further, in the event that reinforcing structures are not isotropic it is desirable to indicate this anisotropy by marking the construct to indicate directionality. This can be accomplished by embedding one or more indicators, such as dyed markings or dyed threads, within the woven reinforcements. The direction or orientation of the indicator will indicate to a surgeon the dimension of the implant in which physical properties are superior to those of other orientations.

As noted above, the manner in which the polymer solution is added to the mold prior to lyophilization helps contribute to the creation of a tissue implant with adequate mechanical integrity. Assuming that a mesh reinforcing material will be used, and that it will be positioned between two thin (e. g., 0.75 mm) shims it should be positioned in a substantially flat orientation at a desired depth in the mold. The polymer solution is poured in a way that allows air bubbles to escape from between the layers of the foam component. Preferably, the mold is tilted at a desired angle and pouring is effected at a controlled rate to best prevent bubble formation. One of ordinary skill in the art will appreciate that a number of variables will control the tilt angle and pour rate. Generally, the mold should be tilted at an angle of greater than about 1 degree to avoid bubble formation. In addition, the rate of pouring should be slow enough to enable any air bubbles to escape from the mold, rather than to be trapped in the mold. If a mesh material is used as the reinforcing component, the density of the mesh openings is an important factor in the formation of a resulting tissue implant with the desired mechanical properties. A low density, or open knitted mesh material, is preferred. One preferred material is a 90:10 copolymer of glycolide and lactide, sold under the tradename VICRYL. One exemplary low density, open knitted mesh is Knitted VICRYL VKM-M. Other preferred materials are polydioxanone or 95:5 copolymer of lactide and glycolide.

The density or “openness” of a mesh material can be evaluated using a digital camera interfaced with a computer. In one evaluation, the density of the mesh was determined using a Nikon SMZ-U Zoom microscope with a Sony digital photo camera DKC-5000 interfaced with an IBM 300PL computer. Digital images of sections of each mesh magnified to 20× were manipulated using Image-Pro Plus 4.0 software in order to determine the mesh density. Once a digital image was captured by the software, the image threshold was set such that the area accounting for the empty spaces in the mesh could be subtracted from the total area of the image. The mesh density was taken to be the percentage of the remaining digital image. Implants with the most desirable mechanical properties were found to be those with a mesh density in the range of about 12 to 80% and more preferably about 45 to 80%.

In one embodiment, the preferred scaffold for kidney repair is a mesh reinforced foam. More preferably, the foam is reinforced with a mesh that includes polydioxanone (PDO) and the foam composition is a copolymer of 35:65 ε-caprolactone and glycolide. The preferred structure to allow cell and tissue ingrowth is one that has an open pore structure and is sized to sufficiently allow cell migration. A suitable pore size is one in which an average diameter is in the range of about 50 to 1000 microns, and more preferably, between about 50 to 500 microns. The mesh layer has a thickness in the range of 1 micron to 1000 microns. Preferably, the foam has a thickness in the range of about 300 microns to 2 mm, and more preferably, between about 500 microns and 1.5 mm. Preferably, the mesh layer has a mesh density in the range of about 12 to 80% and more preferably about 45 to 80%.

In another embodiment, the preferred scaffold for kidney repair is a nonwoven structure. More preferably, the composition of the nonwoven structure is PANACRYL, a 95.5 copolymer of lactide and glycolide, VICRYL, a 90:10 copolymer of glycolide and lactide, or a blend of polydioxanone and VICRYL sold under the tradename ETHISORB. The preferred structure to allow cell and tissue ingrowth is one that has an open pore structure and is sized to sufficiently allow cell migration. A suitable pore size for the nonwoven scaffold is one in which an average diameter is in the range of about 50 to 1000 microns and more preferably between about 100 to 500 microns. The nonwoven scaffold has a thickness between about 300 microns and 2 mm, and more preferably, between about 500 microns and 1.5 mm. The density of the nonwoven can be between 60-150 mg/cc, and more preferably about 60 mg/cc.

Preferred nonwoven materials for scaffold fabrication include flexible, porous structures produced by interlocking layers or networks of fibers, filaments, films, or filamentary structures. Such nonwoven materials can be formed from webs of previously prepared/formed fibers, filaments, or films processed into arranged networks of a desired structure. Generally, nonwoven materials are formed by depositing the constituent components (usually fibers) on a forming or conveying surface. These constituents may be in a dry, wet, quenched, or molten state. Thus, the nonwoven can be in the form of a dry laid, wet laid, or extrusion-based material, or hybrids of these types of nonwovens can be formed. The materials from which the nonwovens can be made are typically polymers, either synthetic or naturally occurring.

Those having ordinary skill in the art will recognize that dry laid scaffolds include those nonwovens formed by garneting, carding, and/or aerodynamically manipulating dry fibers in the dry state. In addition, wet laid nonwovens are well known to be formed from a fiber-containing slurry that is deposited on a surface, such as a moving conveyor. The nonwoven web is formed after removing the aqueous component and drying the fibers. Extrusion-based nonwovens include those formed from spun bond fibers, melt blown fibers, and porous film systems. Hybrids of these nonwovens can be formed by combining one or more layers of different types of nonwovens by a variety of lamination techniques.

In one embodiment, the preferred scaffold for kidney repair is a mesh reinforced foam. More preferably, the foam is reinforced with a mesh that includes polydioxanone (PDO) and the foam composition is a copolymer of 35:65 ε-caprolactone and glycolide. The preferred structure to allow cell and tissue ingrowth is one that has an open pore structure and is sized to sufficiently allow cell migration. A suitable pore size is one in which an average diameter is in the range of about 50 to 1000 microns, and more preferably, between about 50 to 500 microns. The mesh layer has a thickness in the range of about 1 micron to 1000 microns. Preferably, the foam has a thickness in the range of about 300 microns to 2 mm, and more preferably, between about 500 microns and 1.5 mm. In this embodiment, the preferred method of use is to surround the scaffold material with minced tissue. Preferably, the mesh layer has a mesh density in the range of about 12 to 80% and more preferably about 45 to 80%.

In another embodiment, the preferred scaffold for kidney repair is constructed from a polymer that has a slow resorption profile (e.g., at least three months, and preferably, at least six months) and high mechanical strength. Further, the scaffold preferably has a thickness in the range of about 0.5 mm and 5 mm, and more preferably, between about 1 mm and 4 mm. By way of example, the scaffold for ligament repair can include a 95:5 copolymer of lactide and glycolide. In one embodiment, the scaffold for ligament repair can be formed as a composite structure including a 95:5 copolymer of lactide and glycolide and other polymers, such as for example, polylactide, polyglycolide, polydioxanone, polycaprolactone and combinations thereof. The scaffold may be formed of a woven, knit or braided material. Optionally, the polymers from which the scaffold is made can be formed as a nonwoven, textile structure, such as for example, a mesh structure, or alternatively these polymers can be formed as a foam. In another embodiment, the composite structure can include natural polymers, such as for example, collagen, fibrin, or silk. In this embodiment, the natural polymer can act as a coating to the composite structure, or alternatively, the natural polymer can be formed as a foam. The composite structure can also optionally include strips of collagen or silk to reside within the whole scaffold or just the periphery of the scaffold.

One of ordinary skill in the art will appreciate that the selection of a suitable material for forming the biocompatible scaffold of the present invention depends on several factors. These factors include in vivo mechanical performance; cell response to the material in terms of cell attachment, proliferation, migration and differentiation; biocompatibility; and bioabsorption kinetics. Other relevant factors include the chemical composition, spatial distribution of the constituents, the molecular weight of the polymer, and the degree of crystallinity.

The scaffold is preferably provided as a sterile packaged item to be opened at the time of use. The scaffold can be immersed in a solution of saline solution, glucose solution, or culture medium prior to introducing the minced tissue to the scaffold, thereby providing for a more hydrophilic surface as well as providing for the metabolic needs of the cells. The scaffold also can be cut or otherwise shaped to size before use to fit into the defect. Alternatively, multiple layers of about 2-4 mm thick scaffold, each with minced tissues applied on both sides of each layer, can be stacked together to fit into the defect. Biologically active agents, as described above, can also be added to the scaffold before the application of the minced tissue in order to enhance the viability of the cells, or added to the minced tissue prior to the application of the minced tissue to the scaffold. After the application of the minced tissue to the scaffold, fibrin glue, cyanoacrylate adhesive, sutures, or a combination thereof can be used to hold the scaffold in place.

In one embodiment of the present invention a mammal in need of kidney therapy is subject to the removal of a portion of healthy kidney tissue, and the removal, or partial nephrectomy, of a diseased portion of kidney tissue. The cortex and medulla are dissected out from the excised healthy tissue sample and the samples are then minced separately into fine pastes with a scalpel. Using a scalpel, a biocompatible scaffold is shaped in size and contour to match the implant site and the prepared minced tissues are applied separately to opposing surfaces of the scaffold, which is then implanted into the region of the excised, diseased tissue. Additional scaffolds prepared in this manner may be implanted to replace the volume of the diseased tissue removed by partial nephrectomy. The scaffolds are then fixed in place by using sutures, fibrin glue, cyanoacrylate adhesive, or a combination of thereof.

The following specific examples are provided to illustrate the methods and materials of the present invention as they apply to renal therapy. The specific techniques, conditions, materials, proportions and reported data set forth to illustrate the principals and practice of the invention are exemplary and should not be construed as limiting the scope of the invention. Suitable modifications and adaptations of the variety of conditions and parameters normally encountered in surgical situations, which are obvious to those skilled in the art, are within the spirit and scope of the present invention.

### EXAMPLE 1

### Tissue Preparation from Kidney

Healthy kidney tissue samples of approximately 5 cubic mm each were obtained from a porcine source as follows. The kidney tissue was dissected open using a scalpel and tissue was harvested independently from the regions of the cortex and the medulla. The harvested tissues were then rinsed three times in a 50 ml Falcon tube with 5 times the tissue volume with phosphate buffered saline (PBS, Invitrogen, Carlsbad, Calif.). Each wash was for 30 minute duration to remove blood cells before being separately minced in surgical trays by repeatedly chopping and slicing with scalpels until the average particle size was about 500 microns, and no particles were larger than about 1-mm. A section of nonwoven PGA/PLA (90/10) bioresorbable polymer material (Lot # 5213-43-2 from Albany International, Mansfield, Mass.) about 2-mm thick was prepared for use as a scaffold by punching out a 6-mm diameter disc using a core biopsy punch. The scaffold disc was soaked in PBS for 4 hours before use. Using a spatula, the minced cortex tissue was then distributed evenly on one side of the scaffold (˜91 mg per side of a punch) and the minced medulla tissue was then distributed evenly on the second side of the scaffold (˜71 mg per side of a punch). The minced tissues were held tightly in place by applying fibrin glue (from bovine plasma, cat. #46312, Sigma, St Louis, Mo.) liberally over the exterior surfaces of the minced tissue/scaffold device.

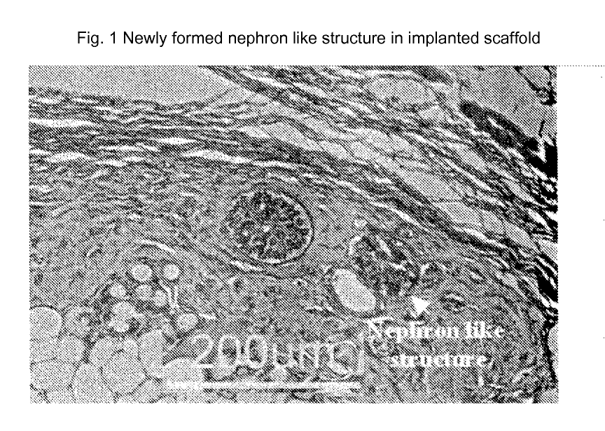

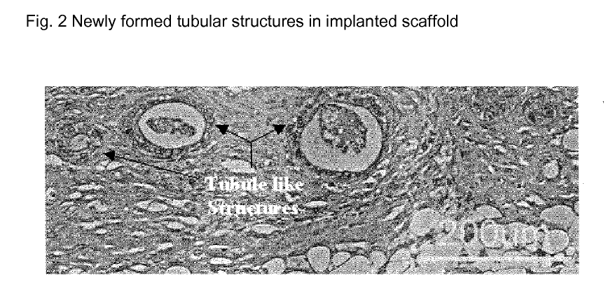

Several such scaffolds loaded with the cortex and medulla tissue pastes were implanted subcutaneously over the lumbar area about 5 mm cranial to the palpated iliac crest on the dorsum, with one on either side of the midline, into SCID mice (Mus Musculus, Fox Chase SCID CB17SC/Male, 5 weeks of age, Taconic Inc. Germantown, N.J.) for 4 weeks, after which they were harvested for examination. Hematoxylin and Eosin (H/E) stained histological sections were analyzed for cell migration, distribution and organization within and around the scaffolds, and for the nature and amount of the matrix formed. Different types of newly formed tubular and immature nephron-like structures were observed, indicating that the multiple types of cells were able to migrate from the minced tissue pastes on the surfaces of the scaffolds and into the scaffold material, and were able to generate renal structural elements. FIG. 1 shows the newly formed nephron like structure and FIG. 2 shows the presence of tubular structures.

### EXAMPLE 2

### Tissue Preparation from an Alternative Tissue Source

Alternatively, one can take a living tissue sample from a site within the body that is not the same as the desired tissue targeted for repair or regeneration and use it to generate the desired target tissue. For example, one can take a tissue sample from epithelium, such as from the salivary gland, skin, liver, lung, etc., mince it, and add to the minced tissue bioactive agents such as drugs, anti-inflammatory agents, proteins, enzymes, growth factors, morphogens, bone morphogenetic proteins, cells, stem cells, progenitor cells, mesenchymal stem cells, embryonic stem cells, renal stem cells, bone marrow aspirate, platelet rich plasma, demineralized collagen, SIS (small intestine submucosa) to ultimately influence the cells in the minced tissue to differentiate or de-differentiate, grow and multiply to develop into a desired tissue type, such as a kidney tissue. The minced tissue with added bioactive agents would be applied to a biocompatible scaffold and immediately implanted into a region of the kidney of a patient to regenerate functional kidney tissue at the implant site. In some embodiments it may be desirable to wait a period of time and allow the cells applied to the scaffold to migrate, differentiate or de-differentiate, grow, multiply, and attach themselves to the scaffold before implanting the scaffold into the recipient host.

### EXAMPLE 3

### Preparation Using Culture Medium

Healthy kidney tissue samples of approximately 5 cubic mm each are obtained separately from the cortex and medulla regions of a kidney. The harvested tissues are placed in separate surgical trays and rinsed with phosphate buffered saline (PBS) and then separately minced until the average particle size is about 500 microns, and no particles are larger than about 1-mm. The size of the tissue particles will vary, but on average should be approximately 500 cubic microns, and no larger than 1 cubic mm. The minced tissues are then distributed uniformly on opposite sides of a synthetic bioresorbable scaffold that has previously been pre-soaked for up to 4 hours in culture medium. The polymer scaffold loaded with minced tissue is then coated with fibrin glue, allowed to cure, and then placed into the medulla of a host kidney in need of renal therapy. The implant is then fixed in place using sutures, with care being taken to ensure intimate contact of the scaffold with the surrounding host kidney tissue.

### EXAMPLE 4

### Preparation Using Bone Marrow Aspirate

Healthy kidney tissue samples of approximately 5 cubic mm each are obtained separately from the cortex and medulla regions of a kidney. The harvested tissues are placed in separate surgical trays and rinsed with phosphate buffered saline (PBS) before being separately minced until the average particle size is about 500 microns, and no particles are larger than about 1-mm. The size of the tissue particles will vary, but on average should be approximately 500 cubic microns, and no larger than about 1 cubic mm. A sample of bone marrow aspirate is obtained from the patient and an aliquot is added to each of the minced tissues. The minced tissues are then distributed uniformly on opposite sides of a synthetic bioresorbable scaffold that has previously been sterilized and pre-soaked in culture medium. The polymer scaffold loaded with minced tissue is then placed into the medulla of a host kidney in need of renal therapy and fixed in place with fibrin glue and resorbable sutures, with care being taken to ensure intimate contact of the loaded scaffold with the surrounding host kidney tissue.

One of ordinary skill in the art will appreciate that it would be useful to have a surgical kit for use in surgery, wherein the kit contains some or components necessary to use and perform the methods of the present invention such as the scaffold and means for mincing the tissue. Preferably the kit is provided in a sterile form suitable for surgical use in the operating room, such as is commonly used in the art.

For example, the kit could have one or more pieces of a polymer scaffold having one or more sizes and shapes, and could further have a plurality of polymer scaffolds having different combinations of sizes and shapes, thereby providing the surgeon with a choice of scaffold sizes and shapes to use.

The kit could also include at least one component selected from the group consisting of fibrin glue, sutures, and cyanoacrylate adhesive, such as would be useful for affixing the polymer scaffold into place. Furthermore, the kit could include one or more surgical scalpels, scissors, forceps, files, rasps, or shavers to be used to mince the tissue and also to shape the scaffold prior to use. The kit could also provide one or more spatulas to used to apply the minced tissue to the scaffold. The kit could also provide one or more tissue biopsy devices, such as a core biopsy needle, to obtain the tissue to be used for mincing.

The kit could also include one or more pharmaceutical and/or bioactive agents to be used according to the methods of the present invention. One or more of the bioactive agents could be lyophilized, and the kit could provide a container of water, preferably sterile water for injection, to reconstitute the bioactive agent. Furthermore, the kit could provide one or more syringes and needles for use in reconstituting the bioactive agents, or for general use and handling of the bioactive agents and minced tissue. In one embodiment, the pharmaceutical and/or bioactive agents are coated onto the scaffold provided to the surgeon.

We claim:

1. A method of treating a mammal in need of renal therapy comprising:
a. removing a sample of kidney tissue
b. separating the cortex and medulla regions of said sample,
c. separately mincing said cortex and said medulla tissue samples,
d. providing a biocompatible polymer scaffold having more than one surface,
e. separately applying said minced cortex and minced medulla tissues to different surfaces of said biocompatible polymer scaffold, and
f. implanting the biocompatible polymer scaffold with said minced tissues into the kidney of said mammal.

2. The method of claim 1 wherein said minced tissues range in size from about 200 microns to about 1 millimeter.

3. The method of claim 1 further comprising pre-soaking said polymer scaffold in culture medium before applying said minced tissues.

4. The method of claim 1 wherein said biocompatible polymer scaffold contains one or more layers of reinforcing material.

5. The method of claim 4 wherein said biocompatible polymer scaffold is a mesh-reinforced foam.

6. The method of claim 1 further comprising the step of coating said polymer scaffold having said minced tissue distributed thereon with fibrin glue.

7. The method of claim 1 further comprising the step of attaching said polymer scaffold to said kidney with sutures.

8. The method of claim 1 further comprising the step of attaching said polymer scaffold to said kidney with cyanoacrylate adhesive.

9. A method of treating a mammal in need of renal therapy comprising:
a. removing a sample of tissue,
b. mincing said tissue sample,
c. adding a bioactive agent top said minced tissue,
d. providing a biocompatible polymer scaffold having a first surface and a second surface,
e. separately applying said minced cortex tissue to said first polymer scaffold surface and applying said minced medulla tissue to said second polymer scaffold surface, and
f. implanting the biocompatible polymer scaffold with said minced tissues into the kidney of said mammal.

10. The method of claim 9 wherein said tissue sample is selected from the group consisting of salivary gland, skin, liver, and lung tissue.

11. The method of claim 9 wherein said bioactive agent is selected from the group consisting of drugs, anti-inflammatory agents, proteins, enzymes, growth factors, morphogens, bone morphogenetic proteins, cells, stem cells, progenitor cells, mesenchymal stem cells, embryonic stem cells, renal stem cells, bone marrow aspirate, platelet rich plasma, demineralized collagen, and small intestine submucosa.

12. A method of treating a mammal in need of renal therapy comprising:
a. removing a sample of kidney tissue,
b. separating the cortex and medulla regions of said sample,
c. separately mincing said cortex and said medulla tissue samples,
d. obtaining a sample of bone marrow aspirate,
e. adding said bone marrow aspirate to each of said minced tissues,
f. providing a biocompatible polymer scaffold having a first surface and a second surface,
g. separately applying said minced cortex tissue to said first polymer scaffold surface and applying said minced medulla tissue to said second polymer scaffold surface, and
h. implanting the biocompatible polymer scaffold with said minced tissues into the kidney of said mammal.

13. The method of claim 12 further comprising the step of coating said polymer scaffold loaded with said minced tissue with fibrin glue.

14. A sterile surgical kit comprised of a tray having a cover, one or more polymer scaffolds, and one or more tissue mincing devices.

15. The surgical kit of claim 14 wherein the tissue mincing device is selected from the group consisting of a scalpel, file, rasp, shaver, scissors, and forceps.

16. The surgical kit of claim 14 further comprising a spatula.

17. The surgical kit of claim 14 further comprising a bioactive agent.

18. The surgical kit of claim 17 wherein the bioactive agent is selected from the group consisting of drugs, anti-inflammatory agents, proteins, enzymes, growth factors, morphogens, bone morphogenetic proteins, cells, stem cells, progenitor cells, mesenchymal stem cells, embryonic stem cells, renal stem cells, bone marrow aspirate, platelet rich plasma, demineralized collagen, and small intestine submucosa.

19. The surgical kit of claim 14 further comprising a container of fibrin glue.

20. The surgical kit of claim 14 further comprising a container of cyanoacrylate adhesive.
